(19) United States
(12) Patent Application Publication
(10) Pub. No.: US 2026/0108701 A1
(43) Pub. Date: Apr. 23, 2026

(54) Chakras Alignment and Healing Device

(71) Applicant: Karlie Johnson, Pittsburgh, PA (US)

(72) Inventor: Karlie Johnson, Pittsburgh, PA (US)

(21) Appl. No.: 19/364,572

(22) Filed: Oct. 21, 2025

Related U.S. Application Data
(60) Provisional application No. 63/710,061, filed on Oct. 22, 2024.

(51) Int. Cl. A61M 21/02; A61H 23/02; A61M 21/00

(52) U.S. Cl. CPC A61M 21/02; A61H 23/0236; A61H 2201/0157; A61H 2201/0192; A61H 2201/5005; A61H 2201/503; A61H 2201/5035; A61H 2201/5048; A61M 2021/0027

(57) ABSTRACT

The present invention is a portable chakra cleansing device to facilitate energetic alignment and healing through sound vibrations and frequencies corresponding to the seven chakras. The device includes a foldable main body for compact storage and portability, a plurality of actuation buttons corresponding to each chakra, and extendable, flexible speaker arms configured to emit chakra-specific sound frequencies. Each speaker arm incorporates a miniature speaker at its free or terminal end to direct sound vibrations toward the intended chakra and is enhanced by embedded copper coils for improved resonance and clarity. The device utilizes an oscillator and microcontroller to generate and store pre-programmed frequencies for each chakra.

### CROSS-REFERENCE TO RELATED APPLICATION

The present application claims priority to, and the benefit of, U.S. Provisional Application No. 63/710,061 which was filed on Oct. 22, 2024 and is incorporated herein by reference in its entirety.

### FIELD OF THE INVENTION

The present invention generally relates to energy healing devices. More specifically, the invention relates to a portable chakra cleansing device designed to facilitate energetic alignment and healing by utilizing sound vibrations and frequencies specific to the seven chakras of the body. The device includes a foldable main body that can be compactly stored and transported. The device includes a plurality of actuation buttons corresponding to each chakra, enabling users to activate pre-programmed sound frequencies in a corresponding extendable and flexible speaker arm for targeted energy alignment. The device further includes a power system with a rechargeable battery, USB port, and retractable power cord. Accordingly, the present disclosure makes specific reference thereto. Nonetheless, it is to be appreciated that aspects of the present invention are also equally applicable to other like applications, devices, and methods of manufacture.

### BACKGROUND

By way of background, sound healing is a form of energy healing and focuses on balancing the mind, body, and spirit. Sound healing origins date back thousands of years, rooted in ancient practices that utilize sound vibrations to promote well-being. The sound healing therapeutic approach is based on the principle that humans are fundamentally composed of energy, and sound, when applied with the correct frequencies (measured in Hz), can align and harmonize the body's energy centers, commonly referred to as chakras or meridian systems.

Energetic alignment achieved through sound therapy alleviates various physical, emotional, and psychological conditions, including pain, anxiety, depression, and high blood pressure. However, when chakras are not cleansed or properly aligned, energy blockages occur, leading to imbalances in the emotional, physical, and spiritual bodies. These misalignments can exacerbate stress and discomfort, impacting overall well-being.

Traditional chakra cleansing methods, while effective, are often time-consuming, costly, and require professional intervention. Traditional chakra cleansing methods create barriers to accessibility for individuals seeking frequent or on-the-go chakra alignment. Individuals desire a quick and cost-effective solution for chakra cleansing and sound-based healing.

Therefore, there exists a long-felt need in the art for a portable chakra cleansing device that provides efficient and precise energetic alignment for the body. There is a long-felt need for a solution that enables individuals to perform chakra cleansing conveniently, without the lengthy and costly processes associated with traditional methods. Additionally, there is a long-felt need for a device that utilizes pre-programmed sound frequencies to align specific chakras effectively. Furthermore, there is a long-felt need for a device that is both customizable and portable, enabling users to perform chakra alignment and sound therapy on-the-go. More specifically, there exists a long-felt need in the art for a foldable, user-friendly chakra cleansing device with flexible speaker arms, intuitive controls, and an affordable design that addresses the limitations of current methods. Finally, there is a long-felt need in the art for a system that easily and effortlessly aligns the meridians/chakras within the body to optimize bodily health and function, as well as emotional and psychological health.

The subject matter disclosed and claimed herein, in one embodiment, comprises an innovative portable chakra cleansing device. The device features a foldable main body designed for compact storage and easy transport. The device includes a plurality of actuation buttons, each corresponding to a specific chakra, and a plurality of extendable and flexible speaker arms that emit chakra-specific sound vibrations. Each speaker arm incorporates a miniature speaker at its free or terminal end to deliver precise vibrational frequencies, while embedded copper coils enhance the resonance and clarity of the sound. The device is equipped with a microcontroller and oscillator for generating and storing pre-programmed frequencies aligned with the seven chakras.

In this manner, the chakra cleansing device of the present invention fulfills the aforementioned needs by offering a portable, efficient, and affordable solution for chakra alignment and energy healing. The device enables users to easily select specific chakras to address, with customizable sound therapy options that can operate individually or simultaneously for holistic healing. The device includes flexible speaker arms, each speaker arm corresponds to one of the actuation buttons, wherein each speaker arm is made of a flexible and durable material. The device is user-friendly, compact, and suitable for use in a variety of settings. The device facilitates energetic alignment and healing by utilizing sound vibrations and frequencies corresponding to the seven chakras of the body

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a portable chakra cleansing device. The device comprises a main body that is foldable along a pivot disposed at the center thereof to enable for compact storage and transport, a plurality of actuation buttons are disposed on a front wall of the main body, wherein each actuation button corresponds to a specific chakra and activates a pre-programmed vibrational frequency associated with the chakra, a plurality of extendable and retractable speaker arms, each corresponding to one of the actuation buttons and designed to emit sound vibrations at the chakra-specific frequency. A miniature speaker is disposed at a free or terminal end of each speaker arm, configured to emit sound vibrations toward the intended chakra for energetic alignment.

In another aspect, each actuation button and corresponding speaker arm are color-coded to visually indicate the associated chakra. The color coding includes red for root chakra, orange for sacral chakra, yellow for solar plexus chakra, green for heart chakra, blue for throat chakra, indigo for third eye chakra, and violet for crown chakra.

In one embodiment, a chakra cleansing device is described. The device includes an oscillator configured to generate pre-programmed chakra-aligned frequencies, a microcontroller is embedded in the device to store and activate the corresponding frequencies when a specific button is pressed, and copper coils are embedded within each speaker arm enhance sound clarity and resonance by amplifying the vibrations generated by the oscillator.

In yet another aspect, the main body includes a plurality of apertures on its rear wall, configured to accommodate clips or hooks for attaching the device to cribs, bed railings, walls, or other surfaces.

In still another embodiment, a method for healing chakras using a portable chakra cleansing device is described. The method comprises activating the chakra cleansing device, the device comprises a main body that includes a plurality of actuation buttons, each corresponding to a specific chakra, a plurality of extendable and retractable speaker arms, each speaker arm corresponding to one of the actuation buttons, a miniature speaker at a free or terminal end of each speaker arm configured to emit sound vibrations at chakra-specific frequencies, selecting one or more chakras to be healed by pressing the corresponding actuation buttons on the device, generating a pre-programmed vibrational frequency for each selected chakra, the frequencies being stored in a microcontroller within the device, extending the speaker arms corresponding to the selected chakras from the main body of the device, positioning the speaker arms over the user's body such that the miniature speakers direct sound vibrations toward the intended chakra points, and emitting the pre-programmed vibrational frequencies from the miniature speakers to stimulate energetic alignment of the selected chakras.

In yet another embodiment, a portable chakra cleansing device is disclosed. The device includes a main body, foldable along a pivot disposed at its center, configured for compact storage and transport, a plurality of actuation buttons, each actuation button corresponds to a specific chakra, a plurality of extendable and retractable speaker arms, each speaker arm corresponds to one of the actuation buttons, wherein each speaker arm is made of a flexible and durable material, a miniature speaker is located at the free or terminal end of each speaker arm, configured to emit sound vibrations at a frequency associated with the corresponding chakra, a power system is integrated into the main body, the power system comprises a rechargeable battery, a retractable power cord, and a USB port for recharging and wired operation.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of chakra cleansing device of the present invention in accordance with the disclosed structure;

FIG. 2 illustrates a schematic view showing internal components used in the chakras cleansing device of the present invention in accordance with the disclosed structure;

FIG. 3 illustrates a perspective view showing the device of the present invention being used with a tripod in accordance with the disclosed structure;

FIG. 4 illustrates a perspective view showing a user using the chakras healing device of the present invention in accordance with the disclosed structure;

FIG. 5 illustrates a partial rear view of the chakras healing device of the present invention in accordance with the disclosed structure; and

FIG. 6 illustrates a perspective view of the chakras healing device of the present invention in a folded state in accordance with the disclosed structure.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a portable chakra cleansing device that provides efficient and precise energetic alignment for the body. There is a long-felt need for a solution that enables individuals to perform chakra cleansing conveniently, without the lengthy and costly processes associated with traditional methods. Additionally, there is a long-felt need for a device that utilizes pre-programmed sound frequencies to align specific chakras effectively. Furthermore, there is a long-felt need for a device that is both customizable and portable, enabling users to perform chakra alignment and sound therapy on-the-go. More specifically, there exists a long-felt need in the art for a foldable, user-friendly chakra cleansing device with flexible speaker arms, intuitive controls, and an affordable design that addresses the limitations of current methods. Finally, there is a long-felt need in the art for a system that easily and effortlessly aligns the meridians/chakras within the body to optimize bodily health and function, as well as emotional and psychological health.

The present invention, in one exemplary embodiment, is a portable chakra cleansing device. The device includes a main body, foldable along a pivot disposed at its center, configured for compact storage and transport, a plurality of actuation buttons, each actuation button corresponds to a specific chakra, a plurality of extendable and retractable speaker arms, each speaker arm corresponds to one of the actuation buttons, wherein each speaker arm is made of a flexible and durable material, a miniature speaker is located at the free or terminal end of each speaker arm, configured to emit sound vibrations at a frequency associated with the corresponding chakra, a power system is integrated into the main body, the power system comprises a rechargeable battery, a retractable power cord, and a USB port for recharging and wired operation.

Reference will now be made in detail to the present preferred embodiments of the invention, examples of which are illustrated in the accompanying drawings. Wherever possible, the same reference numerals are used in the drawings and the description to refer to the same or like parts.

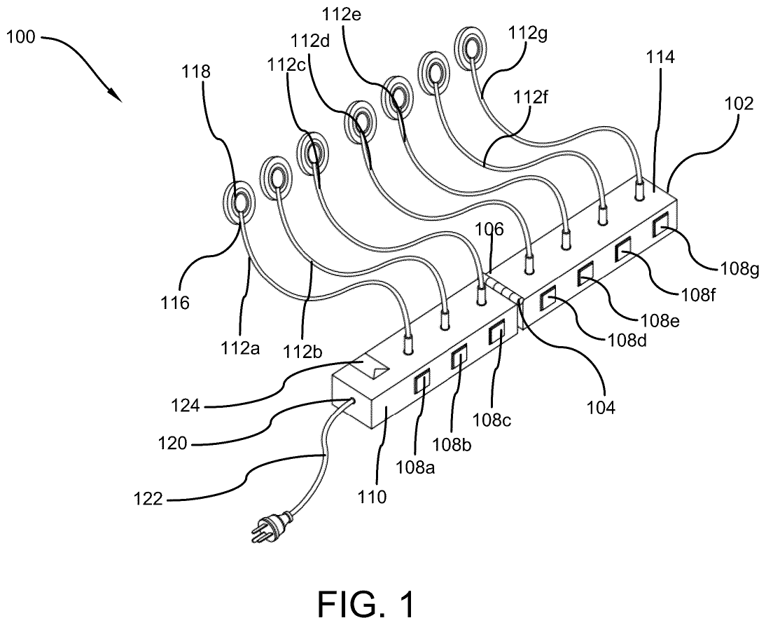

Referring initially to the drawings, FIG. 1 illustrates a perspective view of one potential embodiment of chakra cleansing device of the present invention in accordance with the disclosed structure. The chakra cleansing device 100 of the present invention is designed as a portable, battery-powered device to facilitate energetic alignment and healing by utilizing sound vibrations and frequencies corresponding to the seven chakras of the body. More specifically, the device 100 includes a main body 102 which is preferably cuboidal in shape but can be in any shape and size to accommodate preferences of different users. The main body 102 is foldable and can be folded along a pivot 104 disposed at the center 106 of the main body 102. The foldable construction of the device 100 helps in easy storage and transport of the device 100.

A plurality of actuation buttons 108a-g are disposed on a front wall 110 of the main body 102. Each actuation button of the plurality of actuation buttons 108a-g is configured to activate a pre-programmed frequency sound corresponding to a chakra healing. A plurality of extendable speaker arms 112a-g corresponding to the plurality of actuation buttons 108a-g extend from the top wall 114 of the main body 102. The speaker arms made of a flexible and durable material, such as reinforced silicone or a similar polymer. The arms 112a-g are configured to be retracted or unwound from the main body 102 when in use and can be manually pulled out by a Root Chakra and activates a vibrational frequency aligned with the Root Chakra in the corresponding speaker arm 112a. The vibrational frequency emitted from the speaker 112a has a frequency of 369 Hz. The push button 108b when pushed activates the speaker arm 112b to generate vibrations having a frequency of 417 Hz for Sacral Chakra. The push button 108c activates the speaker arm 112c when pressed by a user and the speaker arm 112c generates vibrations having a frequency of 741 Hz for Throat Chakra. The push button 108d activates the speaker arm 112d when pressed by a user and the speaker 112d generates vibrations having a frequency of 538 Hz for Solar Plexus Chakra. The push button 108e activates the speaker arm 112e when pressed by a user and the speaker 112e generates vibrations having a frequency of 852 or 144 Hz for third eye Chakra. The push button 108f activates the speaker arm 112f when pressed by a user and the speaker arm 112f generates vibrations having a frequency of 639 Hz for Heart Chakra. The push button 108g activates the speaker arm 112g when pressed by a user and the speaker arm 112g generates vibrations having a frequency of 963 Hz for Crown Chakra.

Each extendable speaker arm of the plurality of the extendable speaker arms 112a-g correspond to a specific chakra and associated frequency. Further, the free or terminal end 116 of each extendable speaker arm includes a speaker 118 to emit sound vibrations directly toward the intended chakra and can be positioned over the appropriate body chakras for direct energy alignment. In the preferred embodiment, each arm is color-matched to the corresponding chakra of the arm. Preferably, the arm 112a is color coded in red color for Root chakra. The arm 112b is color coded in orange color for Sacral chakra, the arm 112c is color coded in blue color for Throat chakra, the arm 112d is color coded in yellow color for solar plexus chakra, the arm 112e is color coded in indigo color for third eye chakra, the arm 112f is color coded in green color for heart chakra, and the arm 112g is color coded in violet color for Crown chakra. Same color is coded on the corresponding buttons 108a-g for providing a relevance and correspondence between the chakras and the buttons.

The arms 112a-g can operate individually or together, enabling customization of the sound therapy experience based on the user's needs. The retractable design of the arms 112a-g enables the arms to fold into the main body 102 for easy transport. The adjustable arms provide tailored alignment and therapy for users focusing on specific chakras or overall energy balancing.

A power input port 120 is disposed in the main body 102 for accommodating a charging cord or a power cord 122. The power cord 122 is preferably retractable and enables for direct charging and operation of the device 100 using a conventional wall mounted power supply. The device 100 also includes at least one battery 124 for providing electric power for operation of the device 100. The battery 124 is rechargeable and can be replaced as per preferences of the users.

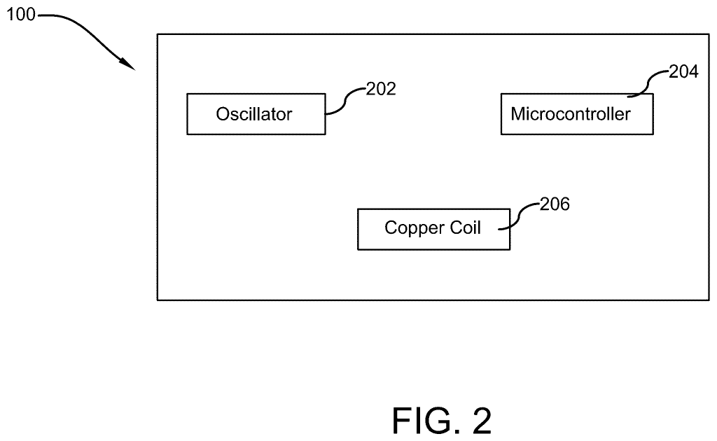

FIG. 2 illustrates a schematic view showing internal components used in the chakras cleansing device of the present invention in accordance with the disclosed structure. The device 100 includes an oscillator 202 which can be in the form of digital wave generator for generating different chakras aligned frequencies. A microcontroller 204 is embedded in device 100 and stores various chakras aligned frequencies. In use, when a specific button is pressed, the microcontroller 204 activates the corresponding frequency for the desired chakra and the oscillator 202 generates the frequency. A copper coil 206 is embedded in each speaker arm (exemplary shown for the speaker arm 112a) and the copper coil 206 enhances the resonance and vibration of the sound of the speaker arm 112a. The copper coils in the speaker arms improve the quality of the sound waves by enhancing their clarity and resonance and the flexibility of the arms enables that the sound waves are directed toward the user's chakra points, thereby increasing effectiveness of the chakras healing.

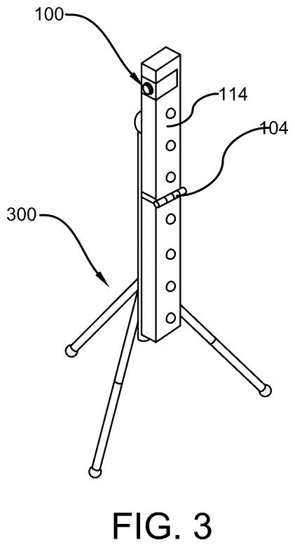

FIG. 3 illustrates a perspective view showing the device of the present invention being used with a tripod in accordance with the disclosed structure. The device 100 is designed to be used with a tripod or any ither similar device for convenient positioning thereof. As illustrated, the device 100 is positioned in a vertical orientation wherein the device 100 is supported by the tripod device 300. The device 100 is positioned such that the top wall 114 is easily accessible and one or more speaker arms can be extended for chakras healing. In the preferred embodiment, the device 100 can be used with any conventional tripod device.

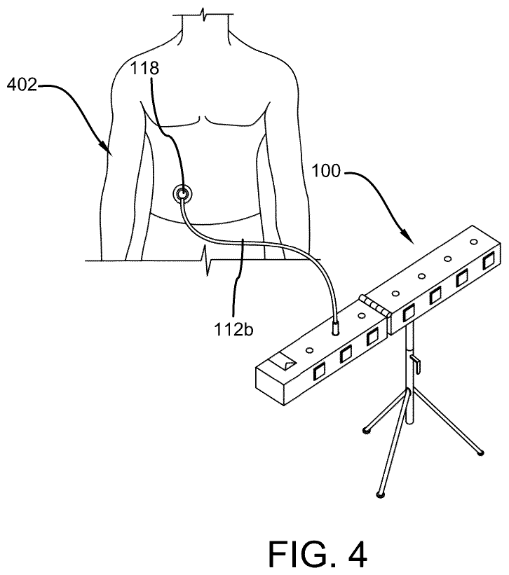

FIG. 4 illustrates a perspective view showing a user using the chakras healing device of the present invention in accordance with the disclosed structure. For healing one or more chakras of the body, a user 402 extends at least one speaker arm from the main body 102. In the present embodiment, as a non-limiting example, the speaker arm 112b is used to generate vibrations having a frequency of 417 Hz for Sacral Chakra. The miniature speaker 118 is adhered to the target body area and the speaker 118 emits vibrations and frequencies that resonate with healing of the Sacral Chakra.

In some embodiments, users can program the device 100 for specific issues, such as stress relief, anxiety, or physical discomfort, using the included meditations and frequency combinations. The sound waves from the speakers work to align the chakras, facilitating improved energy flow and promoting physical, emotional, and psychological well-being.

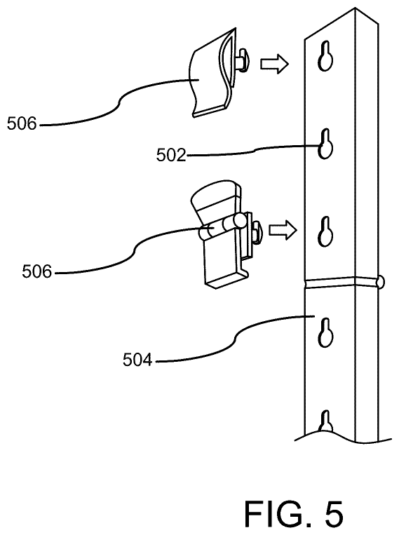

FIG. 5 illustrates a partial rear view of the chakras healing device of the present invention in accordance with the disclosed structure. In the present embodiment, the device 100 has a plurality of apertures 502 disposed on the rear wall 504 thereof. The apertures 502 are designed to accommodate and secure clips 506 or any other hooks for attaching the device 100 to cribs, bed railings, branches, walls, and more. The clips 506 can be integrated or can also be removably attached to the apertures 502.

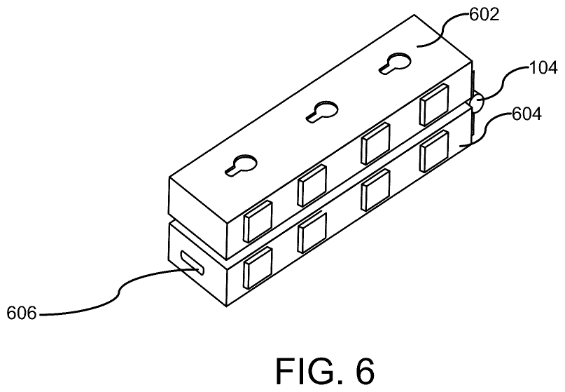

FIG. 6 illustrates a perspective view of the chakras healing device of the present invention in a folded state in accordance with the disclosed structure. The main body 102 is adapted to fold along the pivot 104, thereby enabling the main body 102 to fold not substantially equal sized portions 602, 604, wherein the portions 602, 604 adhere to each other, thereby reducing the length of the main body to half of the extended length (as shown in FIG. 1). A USB port 606 is also provided in the main body 102 for enabling recharging using a conventional USB cord.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “portable chakra cleansing device”, “foldable chakra alignment and healing device”, “chakras healing device”, “chakra cleansing device”, and “device” are interchangeable and refer to the chakra alignment and healing device 100 of the present invention.

Notwithstanding the forgoing, the chakra alignment and healing device 100 of the present invention can be of any suitable configuration as is known in the art without affecting the overall concept of the invention, provided that it accomplishes the above stated objectives. One of ordinary skill in the art will appreciate that the chakra alignment and healing device 100 as shown in the FIGS. are for illustrative purposes only, and that many other configurations of the chakra alignment and healing device 100 are well within the scope of the present disclosure. Although the dimensions of the chakra alignment and healing device 100 are important design parameters for user convenience, the chakra alignment and healing device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A chakra device for energetic healing of the body comprising:
a portable chakra device;
a main body;
a plurality of actuation buttons; and
a plurality of extendable speaker arms;
wherein said portable chakra device having a plurality of sound vibrations and a plurality of sound frequencies corresponding to a plurality of chakras of the body;
wherein each of said plurality of actuation buttons corresponding to one of said plurality of chakras of the body;
wherein each of said plurality of actuation buttons activates a pre-programmed sound vibration and a pre-programmed sound frequency; and
further wherein each of said plurality of extendable speaker arms corresponding to one of said plurality of actuation buttons.

2. The chakra device for energetic healing of the body of claim 1, wherein said plurality of actuation buttons activates seven pre-programmed sound vibrations and seven pre-programmed sound frequencies.

3. The chakra device for energetic healing of the body of claim 2, wherein each one of said seven pre-programmed sound frequencies having a different hertz frequency.

4. The chakra device for energetic healing of the body of claim 3, wherein said seven pre-programmed sound frequencies having a first frequency of 369 Hz, a second frequency of 417 Hz, a third frequency of 741 Hz, a fourth frequency of 538 Hz, a fifth frequency of 852 Hz, a sixth frequency of 639 Hz, and a seventh frequency of 963 Hz.

5. The chakra device for energetic healing of the body of claim 4, wherein said plurality of extendable speaker arms having a flexible material selected from the group consisting of a silicone and a polymer.

6. The chakra device for energetic healing of the body of claim 5, wherein said first frequency aligned with a Root Chakra in a first extendable speaker arm, said second frequency aligned with a Sacral Chakra in a second extendable speaker arm, said third frequency aligned with a Throat Chakra in a third extendable speaker arm, said fourth frequency aligned with a Solar Plexus Chakra in a fourth extendable speaker arm, said fifth frequency aligned with a Third Eye Chakra in a fifth extendable speaker arm, said sixth frequency aligned with a Heart Chakra in a sixth extendable speaker arm, and said seventh frequency aligned with a Crown Chakra in a seventh extendable speaker arm.

7. The chakra device for energetic healing of the body of claim 6, wherein said main body is cuboidal in shape.

8. The chakra device for energetic healing of the body of claim 7, wherein said main body is foldable along a pivot disposed at a center of said main body.

9. The chakra device for energetic healing of the body of claim 3, wherein each of said plurality of extendable speaker arms having a speaker at a terminal end for emitting one of said plurality of sound frequencies.

10. A chakra device for energetic healing of the body comprising:
a portable chakra device;
a main body;
a plurality of actuation buttons; and
a plurality of extendable speaker arms;
wherein said portable chakra device having a plurality of sound vibrations and a plurality of sound frequencies corresponding to a plurality of chakras of the body;
wherein each of said plurality of actuation buttons corresponding to one of said plurality of chakras of the body;
wherein each of said plurality of actuation buttons activates a pre-programmed sound vibration and a pre-programmed sound frequency;
wherein each of said plurality of extendable speaker arms corresponding to one of said plurality of actuation buttons;
wherein said plurality of actuation buttons activates seven pre-programmed sound vibrations and seven pre-programmed sound frequencies; and
further wherein said seven pre-programmed sound frequencies having a first frequency aligned with a Root Chakra in a first extendable speaker arm, a second frequency aligned with a Sacral Chakra in a second extendable speaker arm, a third frequency aligned with a Throat Chakra in a third extendable speaker arm, a fourth frequency aligned with a Solar Plexus Chakra in a fourth extendable speaker arm, a fifth frequency aligned with a Third Eye Chakra in a fifth extendable speaker arm, a sixth frequency aligned with a Heart Chakra in a sixth extendable speaker arm, and a seventh frequency aligned with a Crown Chakra in a seventh extendable speaker arm.

11. The chakra device for energetic healing of the body of claim 10, wherein each one of said seven pre-programmed sound frequencies having a different hertz frequency.

12. The chakra device for energetic healing of the body of claim 11, wherein said seven pre-programmed sound frequencies having said first frequency of 369 Hz, said second frequency of 417 Hz, said third frequency of 741 Hz, said fourth frequency of 538 Hz, said fifth frequency of 852 Hz, said sixth frequency of 639 Hz, and said seventh frequency of 963 Hz.

13. The chakra device for energetic healing of the body of claim 12, wherein said plurality of extendable speaker arms having a flexible material selected from the group consisting of a silicone and a polymer.

14. The chakra device for energetic healing of the body of claim 13, wherein each of said plurality of extendable speaker arms having a speaker at a terminal end for emitting one of said plurality of sound frequencies.

15. A chakra device for energetic healing of the body comprising:
a portable chakra device;
a main body;
a plurality of actuation buttons;
a plurality of extendable speaker arms;
an oscillator; and
a microcontroller;
wherein said portable chakra device having a plurality of sound vibrations and a plurality of sound frequencies corresponding to a plurality of chakras of the body;
wherein each of said plurality of actuation buttons corresponding to one of said plurality of chakras of the body;
wherein each of said plurality of actuation buttons activates a pre-programmed sound vibration and a pre-programmed sound frequency;
wherein each of said plurality of extendable speaker arms corresponding to one of said plurality of actuation buttons;
wherein said oscillator having a digital wave generator for generating said plurality of sound frequencies; and
further wherein said microcontroller aligns each one of said plurality of sound frequencies to an individual one of said plurality of chakras of the body.

16. The chakra device for energetic healing of the body of claim 15, wherein said plurality of actuation buttons activates seven pre-programmed sound vibrations and seven pre-programmed sound frequencies.

17. The chakra device for energetic healing of the body of claim 16, wherein said seven pre-programmed sound frequencies having a first frequency aligned with a Root Chakra in a first extendable speaker arm, a second frequency aligned with a Sacral Chakra in a second extendable speaker arm, a third frequency aligned with a Throat Chakra in a third extendable speaker arm, a fourth frequency aligned with a Solar Plexus Chakra in a fourth extendable speaker arm, a fifth frequency aligned with a Third Eye Chakra in a fifth extendable speaker arm, a sixth frequency aligned with a Heart Chakra in a sixth extendable speaker arm, and a seventh frequency aligned with a Crown Chakra in a seventh extendable speaker arm.

18. The chakra device for energetic healing of the body of claim 17, wherein each one of said seven pre-programmed sound frequencies having a different hertz frequency.

19. The chakra device for energetic healing of the body of claim 18, wherein said seven pre-programmed sound frequencies having said first frequency of 369 Hz, said second frequency of 417 Hz, said third frequency of 741 Hz, said fourth frequency of 538 Hz, said fifth frequency of 852 Hz, said sixth frequency of 639 Hz, and said seventh frequency of 963 Hz.

20. The chakra device for energetic healing of the body of claim 19, wherein each of said plurality of extendable speaker arms having a speaker at a terminal end for emitting one of said plurality of sound frequencies.
